(19) United States
(12) Patent Application Publication
(10) Pub. No.: US 2014/0098939 A1
(43) Pub. Date: Apr. 10, 2014

(54) MOBILE RADIATION GENERATION APPARATUS AND MOBILE RADIATION IMAGING SYSTEM

(71) Applicant: CANON KABUSHIKI KAISHA, Tokyo (JP)

(72) Inventor: Shinji Yamanaka, Tokyo (JP)

(73) Assignee: CANON KABUSHIKI KAISHA, Tokyo (JP)

(21) Appl. No.: 14/044,126

(22) Filed: Oct. 2, 2013

(30) Foreign Application Priority Data
Oct. 5, 2012 (JP) 2012-223304

(51) Int. Cl. A61B 6/00

(52) U.S. Cl. CPC A61B 6/4405
USPC 378/62; 378/102

(57) ABSTRACT

A mobile radiation generation apparatus includes a cart, a radiation generation unit, a positioning member fixed to the cart and configured to movably support the radiation generation unit, a receiving unit configured to store a radiation imaging unit, the receiving unit extending in a direction toward a lower inside portion of the cart from a predetermined position at a side surface of the cart, a control unit configured to control the mobile radiation generation apparatus, and a generated voltage supplying unit disposed to face the control unit across the receiving unit and configured to supply voltage to the radiation generation unit.

### BACKGROUND

1. Technical Field

The present invention relates to a mobile radiation generation apparatus for use, together with an imaging device, to obtain an image of an object subjected to radiation.

2. Description of the Related Art

There are mobile radiation generation apparatuses or apparatuses called mobile radiography systems that include an X-ray generation apparatus placed on a cart, which are capable of imaging a patient not only in an X-ray radiography room but also at a bedside or the like. Such a mobile radiography system is used, together with an imaging device such as a film for obtaining an image in response to generated radiation, an imaging plate (IP) of computed radiography (CR) or a digital radiation detector. Thus, a mobile radiography system includes a receiving unit to store such an imaging device. For example, Japanese Patent Application Laid-Open No. 2012-105787 discusses a mobile radiography system equipped with a pocket on a side surface of the mobile radiography system to store an imaging device.

However, there are problems including that an imaging device has a certain amount of weight, thus it is a burden for an operator to lift and remove an imaging device from a receiving unit in a direction that is opposite to the direction of gravity. Furthermore, when an operator moves an imaging device along the direction of gravity to put the imaging device into a receiving unit, the imaging device may hit a bottom surface of the receiving unit with considerable force. This problem is significant especially in the case of digital radiation detectors, which are relatively susceptible to impact.

### SUMMARY

According to an aspect of the present invention, a mobile radiation generation apparatus includes a cart, a radiation generation unit, a positioning member fixed to the cart and configured to movably support the radiation generation unit, a receiving unit configured to store a radiation imaging unit, the receiving unit extending in a direction toward a lower inside portion of the cart from a predetermined position at a side surface of the cart, a control unit configured to control the mobile radiation generation apparatus, and a generated voltage supplying unit disposed to face the control unit across the receiving unit and configured to supply voltage to the radiation generation unit.

Further features of the present invention will become apparent from the following description of embodiments with reference to the attached drawings. Each of the embodiments of the present invention described below can be implemented solely or as a combination of a plurality of the embodiments or features thereof where necessary or where the combination of elements or features from individual embodiments in a single embodiment is beneficial.

### BRIEF DESCRIPTION OF THE DRAWINGS

FIG. 1 is a schematic view illustrating a mobile radiation generation apparatus according to an embodiment of the present invention.

FIG. 2 is a schematic view illustrating another example of a mobile radiation generation apparatus according to an embodiment of the present invention.

FIG. 3 is a block diagram illustrating a configuration example of a control system of a mobile radiation generation apparatus according to an embodiment of the present invention.

FIG. 4 is a schematic view illustrating first and second housing members of a mobile radiation generation apparatus according to an embodiment of the present invention that can be separated independently from each other.

### DESCRIPTION OF THE EMBODIMENTS

Various embodiments, features, and aspects of the invention will be described in detail below with reference to the drawings.

The following describes an embodiment using as an example an X-ray imaging system configured to perform imaging by use of X-rays as radiation to obtain an X-ray image as a radiation image.

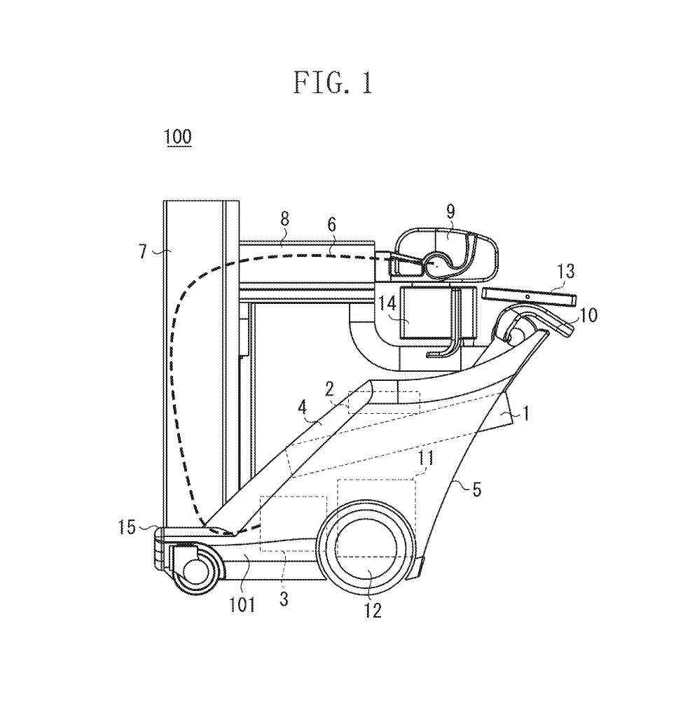

FIGS. 1 to 3 are external views each illustrating an example of a schematic configuration of a mobile X-ray generation apparatus 100 according to the present embodiment. As illustrated in FIG. 1, the mobile X-ray generation apparatus 100 according to the present embodiment includes a cart 101, a radiation generating unit in the form of an X-ray tube 9, a positioning member, and a receiving unit 1. The cart 101 includes wheels 12 so that the cart 101 can move on a floor. The X-ray tube 9 generates X-rays. The positioning member includes a support post portion 7, an arm unit 8 and the like. The positioning member is fixed to the cart 101 and configured to movably support the X-ray tube 9 such that the X-ray tube 9 can be moved. The receiving unit 1 stores an imaging device, which obtains X-ray images in response to X-rays generated from a position determined by the positioning member. When used for bedside imaging and the like at hospitals, the mobile X-ray generation apparatus according to the embodiment and the imaging device are used together as an X-ray imaging system.

In the example illustrated in FIG. 1, the receiving unit 1 extends in a direction toward a lower inside portion of the cart 101 from a side surface of the mobile X-ray generation apparatus that is on the front side in the advancement direction in which the mobile X-ray imaging apparatus is moved forward (to the left in FIG. 1). That is the receiving unit slopes away from the handle 10 at a shallow angle towards the lower front portion of the cart 101. Further, in the example illustrated in FIG. 1, the receiving unit 1 is disposed such that a void portion inside the receiving unit 1 in which the imaging device is to be placed slopes downward with respect to the horizontal direction. Due to this configuration of the receiving unit 1, the imaging device is inserted obliquely along the receiving unit 1 when put into the receiving unit 1. Accordingly, a normal force exerted by a lower side surface of the receiving unit 1 with which the imaging device is in contact cancels a part of the gravity exerted to the imaging device.

Hence, the amount of force that the operator is required to exert on the imaging device in an upward vertical direction (direction that is opposite to the direction of gravity) is smaller than a force exerted on the imaging device by the gravity. Furthermore, since the lower side surface of the receiving unit 1 functions as a guiding member to guide the movement of the imaging device, the operator is required to pay only less attention to the imaging device. Further, when, for example, the imaging device is inserted halfway in the receiving unit 1, even if the operator releases his or her hand from the imaging device, the imaging device will automatically reach a bottom surface portion (back surface portion) of the receiving unit 1 at a rate of acceleration that is definitely lower than that in the case of free fall due to a friction force and a normal force and, consequently, the imaging device will be stored at a most inward portion of the receiving unit 1.

The friction force and the normal force can reduce the impact applied when the imaging device comes into contact with the bottom surface.

When the imaging device is removed from the receiving unit 1, the imaging device is supported by the lower side surface of the receiving unit 1. Thus, the amount of force required to remove the imaging device is smaller than that in the case of lifting the imaging device in the vertical direction. Therefore, the operator can handle the imaging device with ease when storing or removing the imaging device into or from the receiving unit 1.

The foregoing advantageous effects are significant especially in the case in which the imaging device is a digital X-ray imaging unit, as well as in the case in which the imaging device is an analog film cassette or a cassette including an IP of a CR. An example of a digital X-ray imaging unit is a digital X-ray imaging unit including a semiconductor image sensor, which detects X-rays either directly or via a phosphor to obtain electrical signals, and a communication unit, which transfers an X-ray image obtained by the semiconductor image sensor to a mobile radiation generation apparatus in the form of digital data. Such a digital X-ray imaging unit includes many units that are easily affected by impact to be damaged or the like such as the phosphor, the semiconductor image sensor, an amplification unit and an analog-digital (AD) conversion unit for the electric signals, and the communication unit. Therefore, careful handling is required compared with the conventional units. Furthermore, there are digital X-ray imaging units that are configured to have a reduced weight in view of portability and the like, and many of them are less resistant to impact than the film cassettes and the IP cassettes. Meanwhile, it is important to make use of the advantage of immediacy of the digital X-ray imaging unit while maintaining the same level of portability as that of the conventional film cassettes and IP cassettes. When the receiving unit 1 of the mobile radiation generation apparatus according to the embodiment of the present invention is used together with a digital X-ray imaging unit, the operator can enjoy the same level of portability and usefulness as that of the conventional film cassettes and IP cassettes at least when storing or removing the imaging device into or from the receiving unit 1.

The void portion of the receiving unit 1 does not necessarily have to slope. The void portion does not have to slope if a sloping path through which the imaging unit is inserted into or removed from the receiving unit 1 is formed by a plurality of protrusion portions sticking out from side surfaces. However, if the void portion is formed to slope, the configuration of the receiving unit 1 can be simplified to allow for a further reduction in the size of the cart portion.

The angle of the slope of the receiving unit 1, which is a factor that determines the ease of insertion or removal of the imaging device into or from the receiving unit 1, is required to be smaller than 90 degrees when viewed from the horizontal direction. For example, in view of impact on the imaging device, the receiving unit 1 is desirably formed to slope at an angle of 0 to 75 degrees. When the slope of the receiving unit 1 is substantially horizontal, e.g., about 0 to about 30 degrees, an impact applied to the imaging device can be reduced. When the receiving unit 1 slopes at an angle of about 20 to about 40 degrees with respect to the horizontal direction, the operator can remove the imaging device with ease. When the receiving unit 1 slopes at a larger angle, e.g., about 40 to about 75 degrees, the operator can remove the imaging device at a position near the mobile X-ray generation apparatus, compared with a case of removal from the receiving unit 1 sloping at a smaller angle. This is advantageous in that handling becomes easier to improve maneuverability in working spaces.

When the operator using the mobile X-ray imaging system arrives at a desired place, the operator releases his or her hands from the handle 10 and removes the radiation imaging apparatus stored in the receiving unit 1. The receiving unit 1 includes an opening portion immediately below the handle 10 and at a side surface of the cart 101 as illustrated in FIG. 1. Thus, when arriving at a desired place, the operator can remove the imaging device immediately after releasing his or her hands from the handle 10. This improves usability. The opening portion, through which the imaging unit is inserted into or removed from the receiving unit 1, is desirably formed at a position within about 20 cm below the handle 10. Especially when the opening portion of the receiving unit 1 is formed in a side surface of the cart 101 that is opposite to a surface facing the support post portion 7 or along one side surface along the handle 10, the operator can perform operation and removal of the imaging unit more promptly.

In another example, the opening portion of the receiving unit 1 may be formed laterally with respect to the advancement direction of the mobile radiation generation apparatus. This is advantageous when, for example, the advancement direction of the mobile radiation generation apparatus is the longitudinal direction of the mobile radiation generation apparatus, because it is easier to remove the imaging device from a side in view of spaces where the mobile radiation generation apparatus is used. The position of the opening portion can be set at the time of designing or production as appropriate to a situation in which the mobile radiation generation apparatus is to be used.

The handle 10 is provided so that the operator of the mobile radiation generation apparatus can hold the handle 10 to move and drive the cart 101. Although FIG. 1 does not illustrate the handle 10 in detail, the handle 10 has a substantially U-shaped projection along a peripheral portion of a monitor 13 when viewed from above in the vertical direction. The handle 10 is provided along one side surface of the cart 101. In the example illustrated in FIG. 1, the operator is to stand to face the advancement direction (to the left in FIG. 1) in order to operate the handle 10 to move the mobile radiation generation apparatus.

A collimator 14 is coupled to a radiation exit opening of the X-ray tube 9, which is an example of the radiation generation unit, and shaped radiation exits from an exit surface at a lower portion of the collimator 14. A recessed portion is formed to correspond to the collimator 14. The height of the recessed portion in the vertical direction of the cart 101 is reduced with respect to the advancement direction of the mobile radiation generation apparatus. The collimator 14 is accommodated (placed) in the recessed portion. The position at which the collimator 14 is placed may be set as, for example, a non-imaging position (storage position, home position), which is a position of the collimator 14 while the mobile radiation generation apparatus is being moved.

Due to the recessed portion, the X-ray tube 9 and the collimator 14 are disposed at lower positions when they are at their home positions. Thus, the operator's forward field of view is less likely to be blocked by the X-ray tube 9, the collimator 14, or the arm unit 8 to increase all-round visibility. The recessed portion is formed by reducing the height of an upper surface of the cart 101 with which the arm unit 8 is in contact and an upper surface of a monitor fixing member to which the monitor 13 is fixed. Meanwhile, the recessed portion may be configured such that the recessed portion is not U-shaped in a direction that is orthogonal to the advancement direction. Under such a situation, even if, for example, the X-ray tube 9 is at the home position, a support post rotating portion 15 allows the support post portion 7 to be moved without bringing the X-ray tube 9 and the collimator 14 into contact with the cart 101, depending on how the support post rotating portion 15 rotates the support post portion 7. This improves usefulness of the mobile radiation generation apparatus.

A blocking member that absorbs or reflects radiation may be bonded to a bottom surface of the recessed portion to prevent X-rays leaking from the X-ray tube 9 from adversely affecting a control unit or a battery in the cart 101. The blocking member may be formed on an inner side of the bottom surface, i.e., a surface of a member constituting the bottom surface that is on the back surface side of the bottom surface in the cart 101.

As illustrated in FIG. 1, a protruding portion is provided on the exit surface side of the collimator 14. The protruding portion sticks out in an exit direction. The protruding portion comes into contact with, for example, an upper surface of the recessed portion of the cart 101 of the mobile radiation generation apparatus at the non-imaging position. The protruding portion functions as a stopper to reduce the possibility that the exit surface of the collimator 14 comes into contact with the upper surface of the cart 101 and is damaged, etc. For example, the protruding portion may use a member made of an elastic member such as rubber or a shock-absorbing member to reduce impact on the collimator 14 and the upper surface of the cart 101. A plurality of protruding portions may be formed within areas outside areas that are to be irradiated with X-rays from the X-ray generation unit. The protruding portion or a stopper member having the same function as the protruding portion may be provided to the X-ray tube 9, the arm unit 8 and the like.

The arm unit 8 has, for example, a pantograph or telescopic configuration so that the arm unit 8 is extendible and retractable. In the example illustrated in FIG. 1, the arm unit 8 adopts a telescopic configuration. The arm unit 8 is a three-way telescopic arm (extendible and retractable arm with four or more sections) including a first arm unit, a second arm, a third arm, and a fourth arm. The first arm unit is fixed to the support post portion 7 such that the first arm unit can be elevated and lowered. The second arm is movably engaged with the first arm unit. The third arm is movably engaged with the second arm. The fourth arm is movably engaged with the third arm, and the X-ray tube 9 is fixed to one end of the fourth arm. Alternatively, the arm unit 8 may include four or more movable arm sections, for example five, six, or more arm sections. When the maximum length of the arm unit 8 is set to be constant, if the number of arm sections is increased, the minimum length of the arm unit 8 decreases. The maximum length of the arm unit 8 refers to, for example in the case in which the arm unit 8 is a telescopic arm with four or more arm sections, a distance between the support post portion 7 and an end portion of the arm unit 8 at the time when movable second, third, and fourth arm sections are moved to the farthest position from the support post portion 7. The minimum length of the arm unit 8 refers to a distance between the support post portion 7 and an end portion of the arm unit 8 at the time when the second, third, and fourth arm sections are moved to the support post portion 7 as close as possible so that the second, third, and fourth arm sections fit into the first arm section as much as possible. For example, use of a telescopic arm having four arm sections can reduce the minimum length compared with, at least, the case of using an arm with three arm sections. Thus, the telescopic arm having four sections can be stored compactly while the maximum length is increased.

The foregoing configuration also reduces the total size of the mobile radiation generation apparatus while information displayed on the monitor 13 is exposed such that the operator can check the information even when the radiation generation unit is at the home position. Even if the operator does not check information displayed on the monitor 13 while moving the mobile radiation generation apparatus in the positive direction, it is a significant advantage that the operator can check information displayed on the monitor 13 when the X-ray tube 9 is at the home position while, for example, the operator is waiting in front of a patient's room or another medical technologist or the like is positioning a patient. This can significantly improve the usefulness of the apparatus while reducing the size of the apparatus and maintaining the maximum length of the arm unit 8.

The support post portion 7 has multiple sections including a fixed support post portion and a moving support post portion. The fixed support post portion is fixed to the cart 101, and the moving support post portion is fixed to the fixed support post portion such that the moving support post portion can be elevated and lowered. The moving support post portion supports the arm unit 8 via an arm supporting unit and indirectly supports the X-ray tube 9. The fixed support post portion is configured to be rotatable around a shaft of the support post rotating portion 15. The moving support post portion is rotated in response to a rotation of the fixed support post portion so that the X-ray tube 9 can be rotated around a shaft of the support post portion 7.

The support post portion 7 is configured to be extendable and retractable so that when the support post portion 7 is retracted, if, for example, the X-ray tube 9 is at the home position, the height of the support post portion 7 is reduced. Therefore, the support post portion 7 does not block the field of view of the operator to improve forward visibility. This extendable and retractable support post portion 7, together with the telescopic arm having four or more sections, can significantly improve the forward visibility to improve the field of view around the mobile radiation imaging apparatus.

When a digital radiation imaging unit such as a flat panel detector (FPD) is used as the imaging device, the digital radiation imaging unit includes a photoelectric conversion circuit, a phosphor, and a readout circuit. The photoelectric conversion circuit includes a plurality of photoelectric conversion elements arranged to form a matrix and configured to convert radiation into electric signals. The phosphor is deposited on the photoelectric conversion circuit. The readout circuit reads from the photoelectric conversion circuit the electric signals obtained through conversion of X-rays by the phosphor and the photoelectric conversion circuit. When an object is irradiated with X-rays, the phosphor converts the X-rays into visible light, and the photoelectric conversion elements of the photoelectric conversion circuit photoelectrically convert the transmitted X-rays to accumulate a signal charge corresponding to the amount of transmitted X-rays in the photoelectric conversion elements. The readout circuit drives each signal line of the photoelectric conversion circuit to control a switching element connected to the photoelectric conversion elements as appropriate, whereby the readout circuit reads the signal charge accumulated in each of the photoelectric conversion elements one after another as an electric signal, amplifies the electric signal, and then outputs the amplified electric signal. Since the FPD has an advantage that an image can be formed immediately after X-ray imaging, an FPD-mounted mobile radiography system includes an image display unit, an image processing unit and the like as well as an X-ray generation apparatus and the FPD on a single cart.

The mobile X-ray generation apparatus 100 according to the embodiment includes a low-voltage unit 2 and a high-voltage unit 3 as separate units. The low-voltage unit 2 includes a communication unit and a control unit configured to comprehensively control the mobile X-ray generation apparatus 100. The high-voltage unit 3 supplies voltage to the X-ray tube 9. The low-voltage unit 2 controls, for example, conditions and the like under which the X-ray tube 9 generates radiation. The low-voltage unit 2 converts voltage of the battery unit 11 to supply power of a voltage of, for example, 100 V or lower to each unit. On the other hand, the high-voltage unit 3 mainly supplies a voltage required by the X-ray tube 9 to generate X-rays, which is generally a high voltage of several tens of kilovolts. The X-ray tube 9 includes, for example, an electron source, a high-voltage application unit configured to accelerate electrons generated by the electron source, and a target with which the accelerated electrons collide to generate X-rays. When a user presses an irradiation switch (not illustrated) of the mobile X-ray generation apparatus 100, the X-ray tube 9 generates X-rays under predetermined conditions set by the control unit. The high-voltage unit 3 supplies power of a predetermined voltage to a high-voltage application unit. A high-voltage cable 6 runs from the high-voltage unit 3 through the inside or outside of the support post portion 7 and the arm unit 8 to transmit a high voltage to the X-ray tube 9.

As illustrated in FIG. 1, the low-voltage unit 2 and the high-voltage unit 3 are disposed away from each other in the cart 101 to face each other across the receiving unit 1. Therefore, noise caused by the high-voltage unit 3 is attenuated due to the effects of the configuration of the receiving unit 1 and the void portion in the receiving unit 1 so that the noise is less likely to be superimposed on the low-voltage unit 2. Further, since the high-voltage unit 3 is disposed away from the low-voltage unit 2 across the void portion of the receiving unit 1, risks of electrical shock and the like during, for example, the maintenance of the low-voltage unit 2 are reduced to improve maintenance performance. By effectively using the space inside the receiving unit 1, the low-voltage unit 2 and the high-voltage unit 3 can be separated safely from each other without any additional space or blocking member.

In view of the effect of the high-voltage unit 3 on the X-ray imaging device stored in the receiving unit 1, an electromagnetic noise blocking member may be provided to cover the void portion of the receiving unit 1. In this case, the blocking member plays a role in shielding the X-ray imaging device from noise caused by the high-voltage unit 3 and also a role in shielding the low-voltage unit 2 from noise caused by the high-voltage unit 3. Even if such a blocking member is not provided, the high-voltage unit 3 and the low-voltage unit 2 can be separated from each other. Thus, the component costs can be reduced by the cost of the blocking member, and the apparatus size can be reduced.

In the example illustrated in FIG. 1, the high-voltage unit 3 is disposed at a position shifted in the direction of gravity with respect to the void portion of the receiving unit 1 (the high-voltage unit 3 is disposed at a position below the receiving unit 1 in the vertical direction in FIG. 1). Accordingly, the high-voltage unit 3, which generally has a larger weight than the low-voltage unit 2, is disposed at a lower portion of the mobile radiation generation apparatus so that the center of gravity is set to a low position to allow for mechanically stable operation.

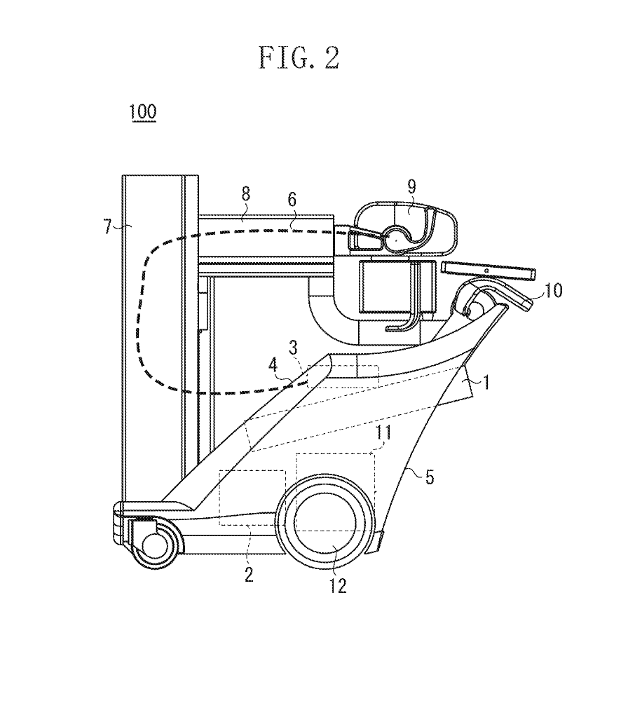

FIG. 2 is a schematic diagram of a mobile X-ray generation apparatus according to another embodiment. A member having a similar configuration to that in FIG. 1 is denoted by the same reference numeral. In the example illustrated in FIG. 2, the high-voltage unit 3 is disposed at a position shifted in a direction that is opposite to the direction of gravity with respect to the void portion of the receiving unit 1 (the high-voltage unit 3 is disposed above the void portion of the receiving unit 1). Accordingly, the high-voltage unit 3 is disposed at a higher position to be close to the X-ray tube 9. This allows the length of the high-voltage cable 6 to be shorter than that in the configuration illustrated in FIG. 1 so that a high voltage can be transmitted effectively to the X-ray tube (radiation generation unit) 9.

In view of the capacity of the battery unit 11, performance of the X-ray tube (radiation generation unit) 9 (maximum tube voltage, tube current, radiation irradiation time), noise-resistance of the radiation imaging apparatus and the like, either one of the arrangement in which the low-voltage power supply unit 2 is disposed above the receiving unit 1 and the high-voltage power supply unit 3 below the receiving unit 1 and the arrangement in which the low-voltage power supply unit 2 is disposed below the receiving unit 1 and the high-voltage power supply unit 3 above the receiving unit 1 can be selected in manufacturing. In the example illustrated in FIG. 2, as in the case described above, the low-voltage unit 2 and the high-voltage unit 3 are spatially separated from each other to maintain a distance between the low-voltage unit 2 and the high-voltage unit 3. Thus, the effect of electromagnetic noise generated by the high-voltage unit 3 on the low-voltage unit 2 can be reduced.

The operator holds the handle 10 to drive the wheels 12 so that the mobile radiation imaging apparatus 100 is moved to a desired place. The wheels 12 are driven in response to an operation of the handle 10. When the handle 10 is not operated, the wheels 12 are in a fixed state. The wheels 12 may be released from the fixed state by operation of the handle 10 either mechanically or electrically.

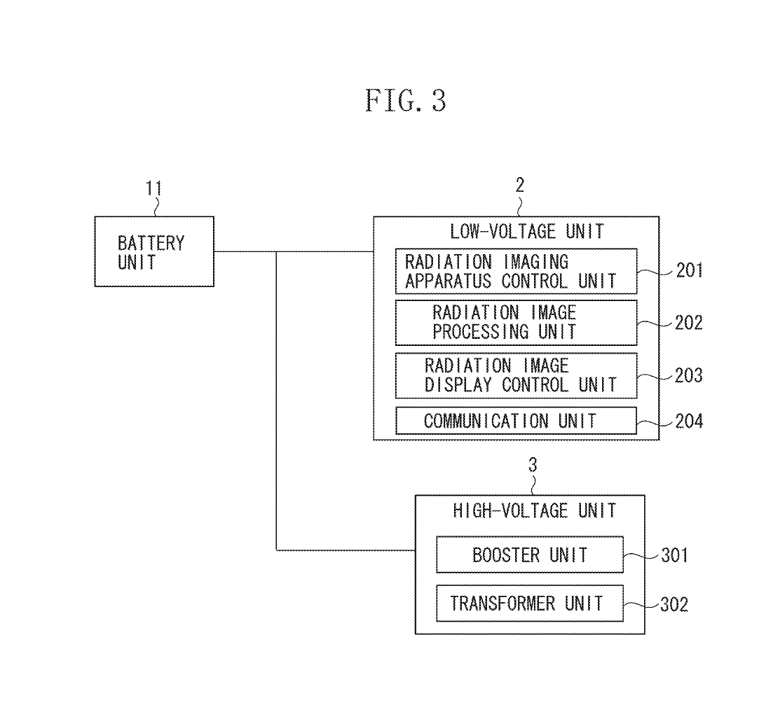

The following describes an example of a control system unit stored in the cart 101, with reference to FIG. 3.

In the example illustrated in FIG. 3, the battery unit 11, which is chargeable by an external input and supplies power to the entire apparatus, is electrically connected to the low-voltage unit 2 including the control unit and the high-voltage unit 3 configured to obtain high voltage for generating X-rays. The high-voltage unit 3 includes a booster unit 301 and a transformer unit 302. When power is supplied from the battery unit 11 to the booster unit 301 and the transformer unit 302, the booster unit 301 and the transformer unit 302 generate high voltage to be supplied to the X-ray generation unit and used to generate X-rays. The booster unit 301 includes, for example, a DC/DC converter to boost direct current (DC) from the battery unit 11 in the form of DC. An inverter converts the boosted DC voltage into alternating current (AC). The transformer unit 302 includes, for example, a transformer to boost AC in the form of AC. High voltage for generating X-rays can be generated without the booster unit 301. However, when the booster unit 301 is provided, a difference between an input voltage and an output voltage of the inverter becomes small. This is efficient and makes designing easy.

The low-voltage unit 2 includes control units such as a radiation imaging apparatus control unit 201, a radiation image processing unit 202, and a radiation image display control unit 203. The radiation imaging apparatus control unit 201 controls driving of the digital X-ray imaging unit. The radiation image processing unit 202 processes X-ray images obtained from the digital X-ray imaging unit and the like. The radiation image display control unit 203 displays processed and/or unprocessed images on the monitor 13. The low-voltage unit 2 also includes a communication unit 204 configured to conduct wireless and/or wired communication with at least one of the radiation imaging unit and an external server. The low-voltage unit 2 may also include an operation control unit configured to detect an input from an operation unit, via which an operation input is received, and control the operation unit, such as a touch-panel position detection device provided integrally with the monitor 13.

In such a control system, the communication unit 204 and the high-voltage unit 3 are disposed to face each other across the void portion of the receiving unit 1. Thus, noise caused by the high-voltage unit 3 is prevented from being superimposed on sent or received information.

The communication unit 204 includes both a wireless communication unit and a wired communication unit to use appropriate one of them as necessary. Further, a communication module configured to communicate with the digital X-ray imaging unit and a communication module configured to communicate with a network in a hospital including a radiology information server (RIS) configured to distribute imaging order information, picture archiving and communication systems (PACS) configured to manage images and the like are provided as separate modules to improve the level of security.

When the communication unit 204 includes a communication control unit configured to control sent or received data and an antenna configured to send or receive data, the antenna is disposed on the side closer to a housing wall of the cart 101 than to the communication unit 204 so that the intensity of radio waves input or output through the antenna can be increased. In the example described above, it is desirable to dispose the antenna along a side surface portion of the cart 101 that is considered to be less shielded when the X-ray tube 9 is at the home position or when imaging is performed.

In the examples illustrated in FIGS. 1 and 2, the battery 11 is stored in a space below the void portion of the receiving unit 1. Since a large quantity of power is generally needed to generate X-rays, the battery 11 tends to be large and heavy. Therefore, the battery 11 is stored in a lower space to increase stability.

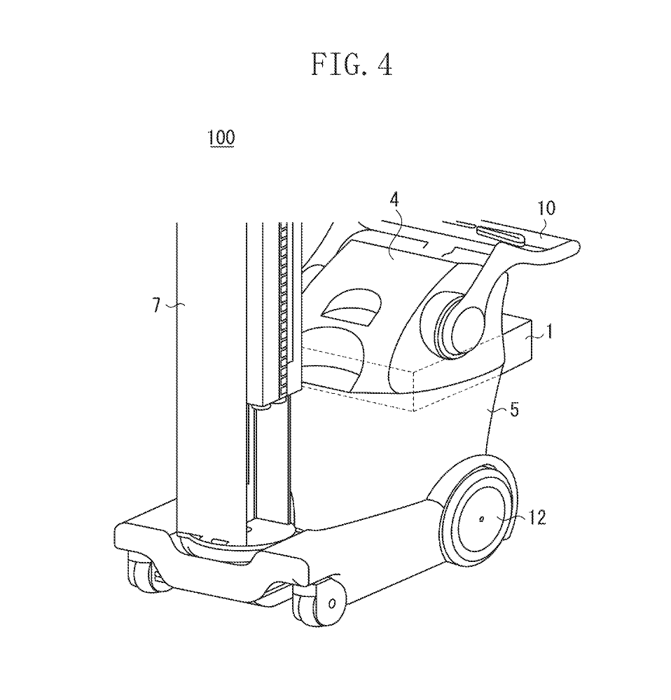

The following describes an external housing and the like that form the space in the cart 101 where the receiving unit 1 and other members are disposed, with reference to FIG. 4.

The external housing of the cart 101 includes a first housing member 4 and a second housing member 5, which are separated near the receiving unit 1. The first housing member 4 is disposed from a middle part to an upper part of side surfaces of the receiving unit 1 to constitute an upper surface of the cart 101. The first housing member 4 forms a space above the receiving unit 1. The first housing member 4 includes a base portion of the handle 10. Thus, the first housing member 4 may include a plurality of members that are separable near the base portion of the handle 10 so that the first housing member 4 can be separated from the cart 101 without removal of the handle 10.

The second housing member 5 is disposed from a middle part to a lower part of the side surfaces of the receiving unit 1. Together with an upper surface member of a bottom portion of the cart 101, which rotatably holds a shaft and the like, the second housing member 5 forms a space below the receiving unit 1. The receiving unit 1 is configured to substantially divide the space inside the housing so that the noise reduction properties and maintenance performance are further improved. In view of a plurality of demands such as noise reduction properties, maintenance performance, and size reduction, the upper and lower spaces may be connected partially via a cross-sectional area of, for example, 5% or smaller of the area of the receiving unit 1.

The first housing member 4 and the second housing member 5 can be attached to or detached from the cart 101 independently from each other without attaching or detaching the other housing member to or from the cart 101. Thus, at the time of inspection of the high-voltage power supply unit 3, which in general requires frequent maintenance, it is required to remove only the first housing 4 (FIG. 1) or the second housing 5 (FIG. 2). This can reduce the amount of work required to conduct the maintenance.

At the time of inspection of the low-voltage power supply unit 2, it is required to remove only the second housing 5 (FIG. 1) or the first housing 4 (FIG. 2) to conduct maintenance. Furthermore, since the low-voltage power supply unit 2 and the high-voltage power supply unit 3 are spatially separated by the receiving unit 1, a maintenance technologist can work without considering risks of touching the high-voltage power supply unit 3 with his hand, etc. This increases the degree of freedom of operation and reduces the operation time.

Especially in the mobile X-ray generation apparatus 100, the housing is a major massive component. In view of safety, it is necessary to conduct maintenance or remove the massive component to repair or exchange inner components in a case of a breakdown. Furthermore, since high voltages are needed to generate X-rays, the low-voltage unit and the high-voltage unit are provided on the same cart. Thus, there have been risks that, for example, a technologist is required to operate a portion near the high-voltage unit when repairing the low-voltage unit. The mobile radiation generation apparatus according to the embodiment overcomes the problems that arise in the above cases to allow for safe maintenance.

In the embodiment illustrated in FIG. 4, the first housing member 4 and the second housing member 5, each of which forms the space inside the cart 101, are configured to be removable. However, the present invention is not limited to this configuration, and an access cover with a surface that is large enough to allow for access to inner units may be provided. For example, the access cover may constitute a door member having one end hinged to at least one of the first housing member 4 and the second housing member 5 so that the door member can be opened or closed. The access cover is to be removed to access units disposed inside the cart 101 to remove the units or conduct maintenance. Opening or removing the access cover refers to both partial removal by opening or closing the door configuration and complete removal by separating from the housing. In any case, the access cover allows for access to the spaces inside the housing.

The access cover can be provided as appropriate to correspond to the arrangement described in the above embodiment. For example, the access cover is disposed at a position corresponding to the battery 11 as a component constituting the second housing member 5. The access cover forms a surface that is larger than at least a side surface of the battery 11 so that the battery 11 can be attached or detached when the access cover is removed or opened. This allows for efficient periodic replacement of the battery 11 by opening or closing the access cover to improve maintenance performance.

Further, for example, the access cover may be provided as a component constituting the first housing member 4 at a position corresponding to the low-voltage unit 2 disposed in an inside space above the receiving unit 1. The size of the access cover is determined such that the low-voltage unit 2 can be visually inspected or attached or detached when the access cover is removed or opened. This enables a technologist to conduct maintenance of the low-voltage unit 2 with ease by removing the access cover. Furthermore, a lot of effort required to remove a large component constituting the housing can be reduced.

The access cover can also be provided to, for example, the upper surface side of the housing of the cart 101.

In the above example, the housing members or the access cover and the arrangement of the receiving unit 1 allow a technologist to open the housing with ease to inspect inner components at the time of maintenance. Furthermore, the risks of conducting an operation near the high-voltage unit 3 during an inspection of the low-voltage unit 2 can be eliminated.

In the mobile X-ray generation apparatus 100 according to the embodiment illustrated in FIG. 4, the movable support post is moved upward with respect to the fixed support post of the support post portion 7, which is rotatably fixed by the support post rotating portion 15, based on an assumption of, for example, the time of use or maintenance. The movable support post is rotated rightward by substantially 90 degrees with respect to the advancement direction. The arm unit 8 and the X-ray tube 9 are disposed away from the cart 101.

Exteriors of the first housing 4, the second housing 5, and the receiving unit 1 are desirably made of an insulating material such as fiber reinforced plastics (FRP) to avoid the risk of contact with the high-voltage power supply unit.

According to the foregoing embodiments, usability of an operator who conducts radiation imaging can be improved. Furthermore, the high-voltage unit and the low-voltage unit can be separated from each other without deteriorating maintenance performance to improve maintenance performance.

Although the foregoing example describes the imaging apparatus using X-rays, the embodiments of the present invention are not limited to the imaging apparatus, and imaging apparatuses using other forms of radiation are also encompassed within the scope of the embodiments of the present invention.

Further, the embodiments of the present invention are not limited to the above examples, and the mobile X-ray generation apparatus may be any mobile X-ray generation apparatus having at least one of the following functions: extension and retraction of the arm unit 8; elevating and lowering of the arm unit 8 with respect to the support post portion 7; extension and retraction of the support post portion 7; and rotation.

Although the foregoing describes a beam member in which the support post portion 7 extends in the vertical direction and the arm unit 8 extends in the horizontal direction, the embodiments of the present invention are not limited thereto. The support post portion 7 may be a member extending in a first direction, and the arm unit 8 may be a member extending in a second direction that is different from the first direction. Alternatively, the support post portion 7 may be incorporated in the cart 101.

Any combination of the above embodiments is encompassed within the scope of embodiments of the present invention.

The X-ray tube 9 may use, but is not limited to, a rotating anode reflection type target. The X-ray tube 9 may also adopt a radiation generation unit including a fixed anode transmission type target. In this case, an anode rotation mechanism and the like become unnecessary so that the size can be reduced and the required level of load tolerance of the support member can be reduced. Thus, the arm unit 8 and the support post portion 7 can be thinned to become more compact. Furthermore, at least members other than the radiation generation unit can be manufactured inexpensively.

The control unit realized by cooperation of a program and hardware is also encompassed within the scope of embodiments of the present invention. In an embodiment directed to a program, a program corresponding to the above processing is stored in a receiving unit, and a central processing unit (CPU) of a control unit develops the program in a random access memory (RAM) to execute instructions contained in the program, whereby the embodiment is realized.

According to the above embodiments, removal of the radiation imaging unit is facilitated, and the possibility of application of impact on the radiation imaging unit during storage can be reduced.

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment (s) of the present invention, and by a method performed by the computer of the system or apparatus by, for example, reading out and executing the computer executable instructions from the storage medium to perform the functions of one or more of the above-described embodiment (s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-223304 filed Oct. 5, 2012, which is hereby incorporated by reference herein in its entirety.

What is claimed is:

1. A mobile radiation generation apparatus comprising:
a cart;
a radiation generation unit;
a positioning member fixed to the cart and configured to movably support the radiation generation unit;
a receiving unit configured to store a radiation imaging unit, the receiving unit extending in a direction toward a lower inside portion of the cart from a predetermined position at a side surface of the cart;
a control unit configured to control the mobile radiation generation apparatus; and
a generated voltage supplying unit disposed to face the control unit across the receiving unit and configured to supply voltage to the radiation generation unit.

2. The mobile radiation generation apparatus according to claim 1, wherein the generated voltage supplying unit is disposed at a position above the receiving unit.

3. The mobile radiation generation apparatus according to claim 1, wherein the generated voltage supplying unit is disposed at a position below the receiving unit.

4. The mobile radiation generation apparatus according to claim 1, wherein a battery configured to supply power to each member of the mobile radiation generation apparatus is disposed below a void portion of the receiving unit.

5. The mobile radiation generation apparatus according to claim 1, wherein a void portion of the receiving unit is formed to slope with respect to a horizontal direction.

6. The mobile radiation generation apparatus according to claim 5, wherein the receiving unit is formed to slope at an angle of 20 degrees to 40 degrees with respect to the horizontal direction.

7. The mobile radiation generation apparatus according to claim 1, wherein an opening portion through which the radiation imaging unit is insertable into or removable from the receiving unit is formed in a side surface of the cart.

8. The mobile radiation generation apparatus according to claim 7, wherein the positioning member includes a support post portion fixed to the cart and extending in a vertical direction and an arm unit coupled to the support post portion and configured to support the radiation generation unit, and
wherein the opening portion of the receiving unit is formed in the side surface that is opposite to a side surface that faces the support post portion in the cart.

9. A mobile radiation imaging system comprising:
the mobile radiation generation apparatus according to claim 1; and
a digital radiation imaging unit including a semiconductor image sensor and a communication unit configured to transfer an X-ray image obtained by the semiconductor image sensor to the mobile radiation generation apparatus.
